(19) United States
(12) Patent Application Publication
(10) Pub. No.: US 2022/0183284 A1
(43) Pub. Date: Jun. 16, 2022

(54) SELF-ADHESIVE, ANTIMICROBIAL, HYGIENIC PLASTIC SHEETS

(71) Applicant: JAMES R. SCHWEER, BIRMINGHAM, AL (US)

(72) Inventor: JAMES R. SCHWEER, BIRMINGHAM, AL (US)

(21) Appl. No.: 17/543,662

(22) Filed: Dec. 6, 2021

Related U.S. Application Data
(60) Provisional application No. 63/125,964, filed on Dec. 15, 2020.

(51) Int. Cl. A01N 25/34; A01N 25/24; A01N 59/16; A01N 33/12; A01N 59/00; A01N 31/02; A01P 1/00

(52) U.S. Cl. CPC A01N 25/34; A01N 25/24; A01N 59/16; A01P 1/00; A01N 59/00; A01N 31/02; A01N 33/12

(57) ABSTRACT

The self-adhesive, antimicrobial, hygienic plastic sheets can be in different sizes and shapes and may be transparent, opaque, colorful prints or patterns including game patterns, such as checkerboard, maze, Sudoku, etc. The sheets may have varying sticking and tensile strength, to allow easy removal without damage to the surface. They may be formed from simple plastic material or recycled or regular paper material to lessen environmental impact. They may be provided on a roll, either with or without perforations for easily removing the sheets from the roll. The perforations may define a single size sheet, or may define multiple size sheets. Raised portions with a central opening provide one or more confined areas on the sheets to help keep items in specific location(s). The raised portions may be attached to the top of the sheets, or may be formed integrally with the sheets.

### CROSS-REFERENCE TO RELATED APPLICATION

This application claims the benefit of U.S. Provisional Patent Application No. 63/125,964, filed on Dec. 15, 2020.

### BACKGROUND

### 1. Field

The disclosure of the present patent application relates to personal protection equipment (PPE), and particularly to self-adhesive, antimicrobial, hygienic plastic sheets.

### 2. Description of the Related Art

In general, plastic wraps provide an easy way to cover food or food storage containers. The plastic wrap is generally provided in the form of a roll in a box that includes a cutting mechanism for cutting off desired lengths of the plastic wrap. These plastic wrap products may be useful for some purposes, but they do not operate satisfactorily as hygienic shields as they lack adhesive properties and do not include antimicrobial properties. In addition, the lack of raised surfaces or portions, make these products unable to stabilize objects placed upon them.

Thus, self-adhesive, antimicrobial, hygienic plastic sheets solving the aforementioned problems is desired.

### SUMMARY

The self-adhesive, antimicrobial, hygienic plastic sheets include a number of embodiments. The sheets can be in different sizes, such as a wide version of a sticky-type of plastic sheets that can cover eating areas, such as trays and high chair trays, and is particularly useful in day care and senior centers and hospitals; a 12″×12″ version of a sticky-type of plastic sheets that can cover smaller areas where people place their hands or food and is particularly useful in dining out, group gatherings, and school cafeterias; and a smaller travel version providing a sticky-type of plastic sheet that can cover areas used by children or adults, especially people on the go, and is particularly useful in airplanes, arm rests, and visiting senior centers and hospitals. The sheets may include perforations or may be provided as pre-cut sheets. They may be transparent, opaque, colorful prints, or patterns, including game patterns, such as checkerboard, maze, Sudoku, etc. The sheets may have varying sticking and tensile strengths to allow easy removal without damage to the surface.

The sheets may be formed from simple plastic material, or recycled or regular (non-recycled) paper material (for environmental concerns), and may incorporate anti-microbial and/or otherwise sanitary materials by impregnating or applying the materials throughout the sheets, or only on portions of a sheet. The anti-microbial and/or sanitary materials may include alcohol, benzalkonium chloride, nanotechnology, silver, and baking soda. The sheets may be washable/re-usable or intended for single use (disposable). They may be supplied as one sheet size per box or assorted size options per box, and may have different shapes, such as elongated, square, rectangular, circular, and/or patterned. Raised portions with a central opening may provide one or more confined utility areas on the sheets to help keep food, toys, etc. in specific location(s). The raised portions may be attached to the top of the sheets, or may be formed integrally with the sheets. The sheets can be provided on a roll, either with or without perforations for easily removing the sheets from the roll. The perforations may define a single size sheet, or may define multiple size sheets.

These and other features of the present subject matter will become readily apparent upon further review of the following specification and drawings.

### BRIEF DESCRIPTION OF THE DRAWINGS

FIG. 1 is an environmental, perspective view of a first embodiment of self-adhesive, antimicrobial, hygienic plastic sheets, shown attached to an infant's highchair.

FIG. 2 is top view of the self-adhesive, antimicrobial, hygienic plastic sheets of FIG. 1.

FIG. 3 is a section view along lines 3-3 of FIG. 2.

FIG. 4 is top view of a second embodiment of self-adhesive, antimicrobial, hygienic plastic sheets.

FIG. 5 is a section view along lines 5-5 of FIG. 4.

FIG. 6 is top view of a third embodiment of self-adhesive, antimicrobial, hygienic plastic sheets.

FIG. 7 is a section view along lines 7-7 of FIG. 6.

FIG. 8 is a perspective view of a roll of antimicrobial, hygienic plastic sheets.

Similar reference characters denote corresponding features consistently throughout the attached drawings.

### DETAILED DESCRIPTION OF THE PREFERRED EMBODIMENTS

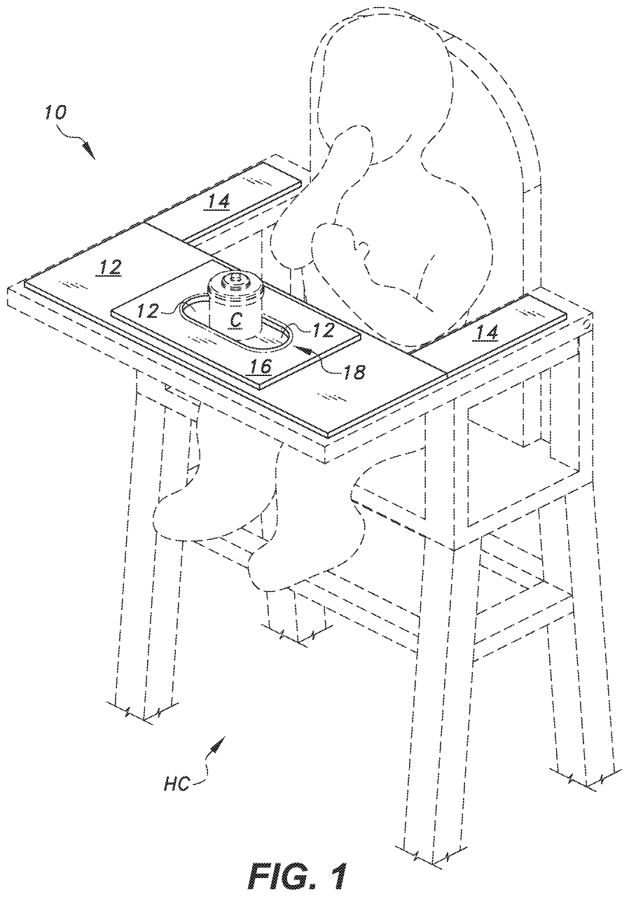

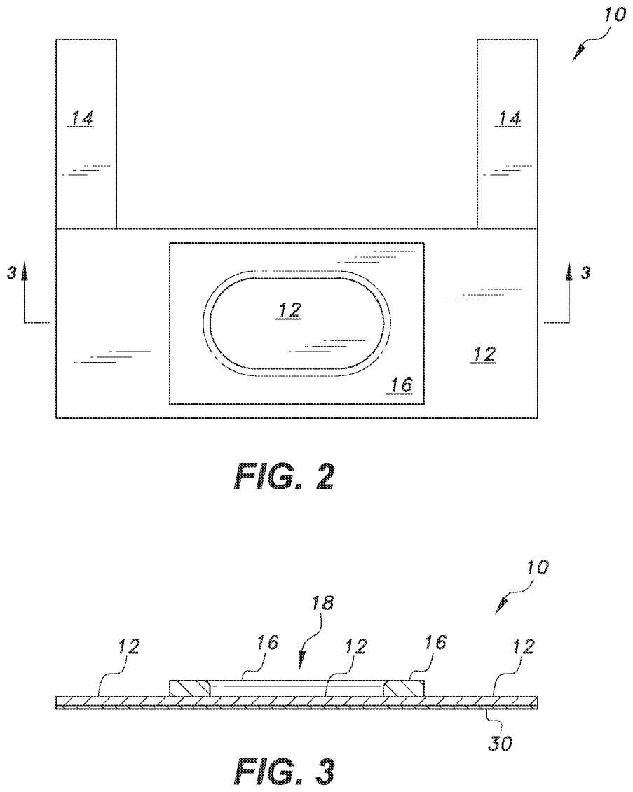

FIG. 1 illustrates one specific use of an embodiment of the self-adhesive, antimicrobial, hygienic plastic sheets 10, wherein the sheets 10 are used to cover the upper surfaces of a highchair HC. As seen in FIGS. 1-3, the sheets include a main body or sheet 12 and auxiliary side strips or sheets 14. The dimensions of the sheets 12, 14 can be varied as described above, but in this embodiment they would be sized to fit the more popular highchairs on the market. A raised portion 16 is placed on top of the main sheet 12 and includes a central opening 18. The raised portion can be made of the same material as the sheets 12, 14, as listed above. The central opening 18 provides a confined area where objects, such as a cup C, can be maintained. An adhesive layer 30 (see FIG. 3) on the bottom surfaces of the sheets 12, 14, provides a light adhesion between the sheets 12, 14 and the upper surface of the highchair's tray and/or arms. The light adhesion holds the sheets 12, 14 in place, while still allowing easy removal of the sheets 12, 14.

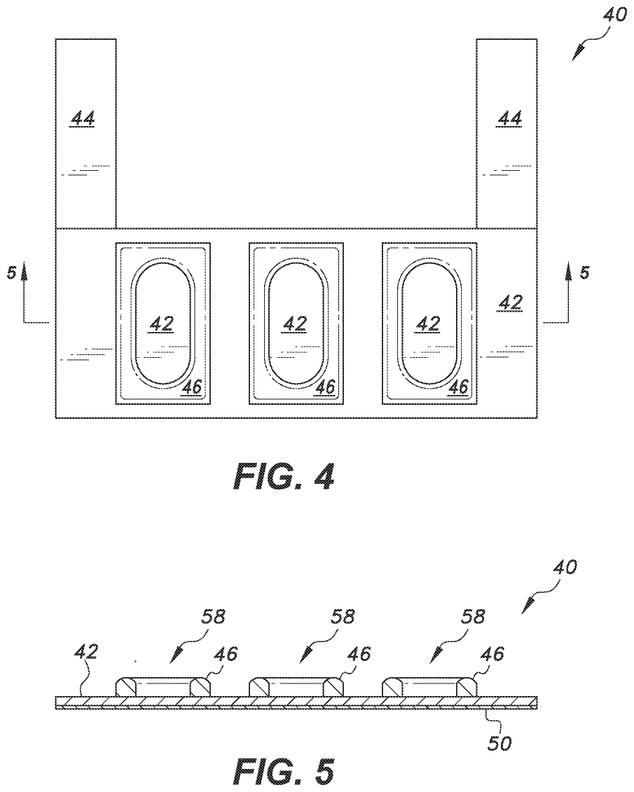

A second embodiment of the self-adhesive, antimicrobial, hygienic plastic sheets 40 is shown in FIGS. 4-5. As in the first embodiment, the sheets 40 include a main body or sheet 42 and auxiliary side strips or sheets 44. The dimensions of the sheets 42, 44 can be varied, as described above. In this embodiment, three raised portions 46 are placed on top of the main sheet 42 and include central openings 58. The raised portions 46 can be made of the same material as the sheets 42, 44, as listed above. The central openings 58 provides confined areas where objects can be maintained. An adhesive layer 50 (see FIG. 5) on the bottom surface of the sheets 42, 44, provides a light adhesion between the sheets 42, 44 and the surface on which they are applied. The light adhesion holds the sheets 42, 44 in place, while still allowing easy removal of the sheets 42, 44.

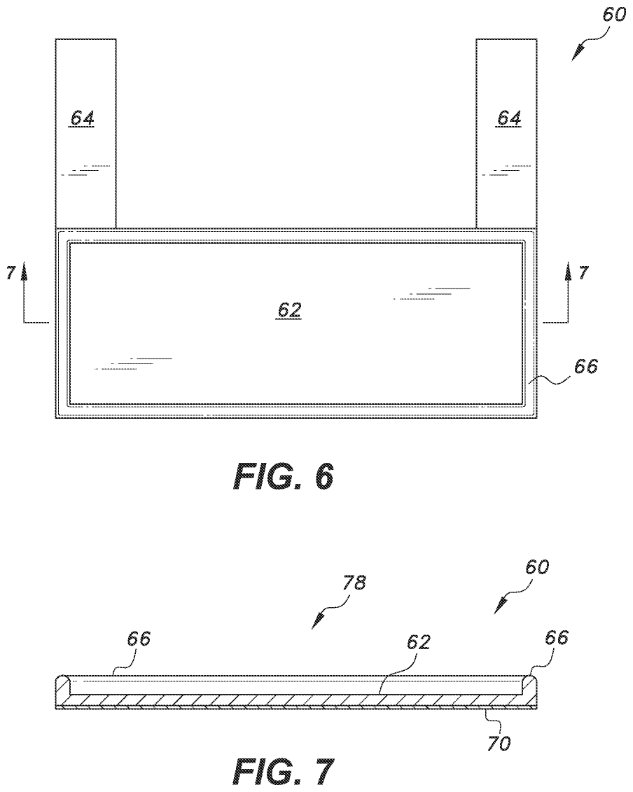

A third embodiment of the self-adhesive, antimicrobial, hygienic plastic sheets 60 is shown in FIGS. 6-7. As in the previous embodiments, the sheets 60 include a main body or sheet 62 and auxiliary side strips or sheets 64. The dimensions of the sheets 62, 64 can be varied as described above. In this embodiment, the raised area 66 is placed on top of and around the perimeter of the main sheet 62, forming a large central opening 78. The raised portion 66 can be made of the same material as the sheets 62, 64, as listed above. The central opening 78 provides a large area for objects to be placed, while the raised portion 66 helps to keep objects from falling off the main sheet 62. An adhesive layer 70 on the bottom surface of the sheets 62, 64, provides a light adhesion between the sheets 62, 64 and the surface on which they are applied. The light adhesion holds the sheets 62, 64 in place, while still allowing easy removal of the sheets 62, 64.

It should be noted that the raised portions 16, 46, 66 may be separate from the sheets 12, 42, 62 as shown in FIGS. 1, 3 and 5, with respect to the first two embodiments, or they may be integral with the sheets, as shown in FIG. 7 with respect to the third embodiment. When they are separate items they may be packaged together or separately. When they are integral, the sheets and integral raised portions can be packaged together, preferably in a stacked arrangement.

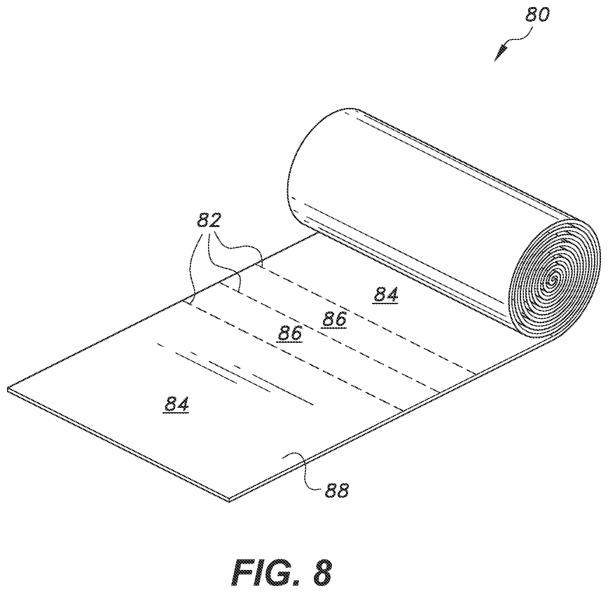

FIG. 8 shows the self-adhesive, antimicrobial, hygienic plastic sheets provided as a roll 80 of plastic sheets. The roll 80 of plastic sheets may be provided as a continuous web, such that the consumer may cut the web (with scissors, for example) to create their own self-adhesive, antimicrobial, hygienic plastic sheets, in the size and shape of the area they wish to cover. Alternatively, the roll 80 of plastic sheets may be provided with perforations 82 extending laterally across the web from one side edge to the other, to allow a consumer to easily tear off individual sheets. While the perforations may be spaced at uniform distances when only a single size of sheet is desired, in the example shown in FIG. 8, the perforations are spaced to provide large sheets 84 and small sheets 86. In this example, the large sheets 84 may correspond to sheets 12, 42, 62 and the small sheets 86 may correspond to sheets 14, 44, 64, in the embodiments described above. The top surface of the web includes an adhesive layer 88 that provides light adhesion, such that the web is easily unrolled from the roll 80.

It is to be understood that the self-adhesive, antimicrobial, hygienic plastic sheets are not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A self-adhesive, antimicrobial, hygienic sheet, comprising:
a main sheet having a top surface and a bottom surface, the main sheet being made of a material being selected from the group consisting of plastic and paper;
a sanitary material, the sanitary material being applied to at least a portion of the main sheet;
an adhesive layer on the bottom surface of the main sheet; and
at least one raised portion on the top surface of the main sheet, the at least one raised portion having an opening defined therein for maintaining an object in a desired area on the top surface of the main sheet.

2. The self-adhesive, antimicrobial, hygienic sheet according to claim 1, wherein the at least one raised portion consists of a single raised portion.

3. The self-adhesive, antimicrobial, hygienic sheet according to claim 2, wherein:
the main sheet defines a perimeter; and
the single raised portion extends around the perimeter of the main sheet.

4. The self-adhesive, antimicrobial, hygienic sheet according to claim 1, wherein the at least one raised portion comprises multiple raised portions.

5. The self-adhesive, antimicrobial, hygienic sheet according to claim 1, wherein the at least one raised portion comprises three raised portions.

6. The self-adhesive, antimicrobial, hygienic sheet according to claim 1, further comprising two auxiliary side sheets, each auxiliary side sheet having a top surface and a bottom surface; each of the auxiliary side sheets having an adhesive layer on the bottom surface thereof.

7. The self-adhesive, antimicrobial, hygienic sheet according to claim 6, wherein the two auxiliary side sheets are made of the same material as the main sheet.

8. The self-adhesive, antimicrobial, hygienic sheet according to claim 7, wherein the main sheet and the two auxiliary side sheets are made of plastic.

9. The self-adhesive, antimicrobial, hygienic sheet according to claim 8, wherein the sanitary material is chosen from the group consisting of alcohol, benzalkonium chloride, nanotechnology, silver, and baking soda.

10. The self-adhesive, antimicrobial, hygienic sheet according to claim 7, wherein the main sheet and the two auxiliary side sheets are made of paper.

11. The self-adhesive, antimicrobial, hygienic sheet according to claim 10, wherein the sanitary material is chosen from the group consisting of alcohol, benzalkonium chloride, nanotechnology, silver, and baking soda.

12. The self-adhesive, antimicrobial, hygienic sheet according to claim 1, wherein the main sheet is made of plastic.

13. The self-adhesive, antimicrobial, hygienic sheet according to claim 12, wherein the sanitary material is chosen from the group consisting of alcohol, benzalkonium chloride, nanotechnology, silver, and baking soda.

14. The self-adhesive, antimicrobial, hygienic sheet according to claim 1, wherein the main sheet is made of paper.

15. The self-adhesive, antimicrobial, hygienic sheet according to claim 14, wherein the sanitary material is chosen from the group consisting of alcohol, benzalkonium chloride, nanotechnology, silver, and baking soda.

16. A continuous web roll of self-adhesive, antimicrobial, hygienic plastic sheets, each of the self-adhesive, antimicrobial, hygienic sheet comprising:
a main sheet having a top surface and a bottom surface, the main sheet being made of a material being selected from the group consisting of plastic and paper;
a sanitary material, the sanitary material being applied to at least a portion of the main sheet;
an adhesive layer on the bottom surface of the main sheet; and
at least one raised portion on the top surface of the main sheet, the at least one raised portion having an opening defined therein for maintaining an object in a desired area on the top surface of the main sheet.

17. The continuous web roll of self-adhesive, antimicrobial, hygienic plastic sheets according to claim 16, further comprising a plurality of perforations, the perforations allowing a consumer to easily tear off individual self-adhesive, antimicrobial, hygienic sheets.

18. The continuous web roll of self-adhesive, antimicrobial, hygienic plastic sheets according to claim 16, wherein the sanitary material is chosen from the group consisting of alcohol, benzalkonium chloride, nanotechnology, silver, and baking soda.

19. The self-adhesive, antimicrobial, hygienic sheet according to claim 18, wherein the main sheet is made of plastic.

20. The self-adhesive, antimicrobial, hygienic sheet according to claim 18, wherein the main sheet is made of paper.
